(19) United States
(12) Patent Application Publication
(10) Pub. No.: US 2026/0166023 A1
(43) Pub. Date: Jun. 18, 2026

(54) METHODS OF TREATING IMMUNOTHERAPY-INDUCED CYTOKINE RELEASE SYNDROME WITH TARGETED RETROVECTORS

(71) Applicant: DeltaNext-Gene, Inc., Santa Monica, CA (US)

(72) Inventors: Erlinda Gordon, Santa Monica, CA (US); Frederick Hall, Carmel Valley, CA (US)

(21) Appl. No.: 19/428,047

(22) Filed: Dec. 19, 2025

Related U.S. Application Data
(63) Continuation-in-part of application No. 17/976,164, filed on Oct. 28, 2022.
(63) Continuation of application No. PCT/US2021/030282, filed on Apr. 30, 2021.

(51) Int. Cl. A61K 31/454; A61K 9/00; A61K 31/496; A61K 40/11; A61K 40/24; A61K 40/31; A61K 40/42

(52) U.S. Cl. CPC A61K 31/454; A61K 9/0019; A61K 31/496; A61K 40/11; A61K 40/24; A61K 40/31; A61K 40/4211

(57) ABSTRACT

The present disclosure relates to methods of administering a chimeric amphotropic RNA vector encoding an anti-cyclin G1 construct or two killer genes (an anti-cyclin G1/HSV-tk) to prevent, treat and/or lessen the effects of cytokine release syndrome (CRS), especially CRS associated with an immunotherapy, for example CAR-T cell therapy.

### CROSS-REFERENCES TO RELATED APPLICATIONS

This application is a continuation-in-part of U.S. patent application Ser. No. 17/976,164, filed on Oct. 28, 2022, entitled “DAMAGE-TARGETED TREATMENT OF DISEASE,” which is a United States National Stage Application under 35 U.S.C. 371 of International Patent Application No. PCT/US2021/030282, filed on Apr. 30, 2021, entitled “DAMAGE-TARGETED TREATMENTS OF DISEASE,” which in turn claims priority to U.S. Provisional Patent Application No. 63/018,298 , filed on Apr. 30, 2020, entitled “METHODS FOR TREATING DISEASES AND DISORDERS,” and U.S. Provisional Patent Application No. 63/078,159 , filed on Sep. 14, 2020, entitled “DAMAGE-TARGETED TREATMENTS OF DISEASE,” each of which is hereby incorporated herein by reference in its entirety.

### BACKGROUND OF THE INVENTION

Chimeric Antigen Receptor-T (CAR-T) cell therapy is an emerging cancer treatment that enhances the patient's immune system to eliminate cancerous cells. Chimeric antigen receptors (CARs) are engineered to recognize and bind antigens specific to the patient's cancer cells. These CARs are subsequently inserted into T cells extracted from the patient, transforming them into CAR-T cells. Once intravenously infused into the patient, the CAR-T cells can target and eliminate the cancer cells. There are currently six United States Food and Drug Administration (USFDA) approved CAR-T cell therapies designed to identify either the Cell Determinant-19 (CD-19) antigen or the B-Cell Maturation Antigen (BCMA), but several factors continue to limit the efficacy of these treatments, including minimal CAR-T cell proliferation and endurance due to an inflammatory tumor microenvironment (TME) and high baseline tumor burden. CAR-T cell therapy has shown significant efficacy in treating certain hematologic malignancies, with several FDA-approved products available as second-line treatments for B-cell lymphoma. However, its administration remains restricted to major medical centers, partly due to the risk of severe acute toxicities requiring intensive care support.

Cytokine Release Syndrome (CRS) is one of the toxicities associated with CAR-T cell therapy. CRS is characterized by rapid and substantial elevations of inflammatory cytokines and immunomodulatory proteins. Cytokines coordinate the immune response, which if excessive, can be severely toxic to the cardiovascular, pulmonary, renal, hematologic, hepatic, and gastrointestinal systems. CRS of varying severity is observed in patients treated with CD19-targeted CAR-T cell therapy. It has also been reported for CAR-T cell therapy targeting HER2, BCMA, and mesothelin. Severe CRS has been recognized as a primary factor contributing to patient fatalities, and its severity has also been correlated with a higher incidence of infectious complications. These observations underscore the critical need for the development of effective strategies for the prevention, diagnosis, and treatment of CRS in patients who have received Chimeric Antigen Receptor T cell (CAR-T) therapy.

One of the core cytokines elevated in CRS patient serum is the inflammatory interleukin-6 (IL-6) produced by monocytes, macrophages, and T cells. Elevated IL-6 levels post-treatment have been correlated with diminished response to CAR-T cell therapy and severe CRS. Other cytokines implicated in CRS include tumor necrosis factor-alpha (TNF-α), interferon-gamma (IFN-γ), interleukin-1 beta (IL-13), and interleukin-10 (IL-10), which contribute to systemic inflammation, endothelial activation, and vascular leakage. The excessive cytokine release can lead to multi-organ dysfunction, hypotension, and, in severe cases, life-threatening complications such as disseminated intravascular coagulation (DIC) and acute respiratory distress syndrome (ARDS).

At present, tocilizumab (ACTEMRA®) and corticosteroids are the most common treatments for CRS, with additional agents such as etanercept and infliximab serving as less frequently used alternatives. Tocilizumab, a neutralizing antibody for interleukin-6 receptor α (IL-6Rα), was first considered as a treatment for CRS based on the observation that patients undergoing CRS after CAR-T cell transfer had dramatically elevated levels of IL-6. In 2017, the administration of tocilizumab became an FDA-approved strategy for CRS management in adoptive T-cell therapy.

Tocilizumab is the standard treatment for mild CRS, with additional steroid therapy being often required in cases of severe CRS. However, the use of corticosteroids can lead to lymphopenia and a reduction in the number of engrafted CAR-T cells.

The present disclosure provides methods of administering a chimeric amphotropic RNA vector (e.g., CAR-V™ vector) encoding an anti-cyclin G1 construct to prevent, treat and/or lessen the effects of cytokine release syndrome (CRS), especially CRS associated with an immunotherapy, for example CAR-T cell therapy.

### BRIEF SUMMARY OF THE INVENTION

The present disclosure provides a method of preventing, treating or lessening the effects of cytokine release syndrome (CRS) or an associated disorder in a patient, the method comprising administering a therapeutically effective amount of a chimeric amphotropic RNA vector (e.g., CAR-V™ vector) encoding an anti-cyclin G1 construct to the patient, wherein the patient has received, is receiving or about to receive an immunotherapy. In some embodiments, the immunotherapy is a Chimeric Antigen Receptor T cell (CAR-T) therapy, an antibody-based therapy, a Natural Killer cell (NK-cell) therapy, an Immune Checkpoint Inhibitors (ICI) therapy, a Tumor-Infiltrating Lymphocytes (TIL) therapy, a cytokine therapy, a Bispecific T-cell Engagers (BiTEs) therapy, a neoTCR-T therapy or any other Adoptive Cell Transfer (ACT) therapy.

In some embodiments, the present disclosure provides a method of preventing, treating, or lessening the effects of CRS or an associated disorder in a patient, the method comprising administering a therapeutically effective amount of a chimeric amphotropic RNA vector (e.g., CAR-V™ vector) encoding an anti-cyclin G1 construct or two killer genes (an anti-cyclin G1/HSV-tk) to the patient.

In some embodiments, the present disclosure provides a method of preventing, treating or lessening the effects of cytokine release syndrome (CRS) or an associated disorder in a patient, the method comprising administering a therapeutically effective amount of a chimeric amphotropic RNA vector (e.g., CAR-V™ vector) encoding either an anti-cyclin G1 construct or two killer genes (anti-cyclin G1 construct and HSV-tk) to the patient, wherein the patient has received, is receiving or about to receive an immunotherapy, wherein the chimeric amphotropic RNA vector (e.g., CAR-V™ vector) is administered before the administration of the immunotherapy, at the same time as the administration of the immunotherapy, and/or after the administration of the immunotherapy.

In some embodiments, the present disclosure provides a method of preventing, treating or lessening the effects of cytokine release syndrome (CRS) or an associated disorder in a patient, the method comprising administering a therapeutically effective amount of a chimeric amphotropic RNA vector (CAR-V™ vector) encoding an anti-cyclin G1 construct or two killer genes (anti-cyclin G1 construct and HSV-tk) to the patient, wherein the patient has received, is receiving or about to receive an immunotherapy, wherein the CAR-V™ vector is administered before the development of CRS, or symptoms thereof, and/or after the development of CRS, or symptoms thereof.

In some embodiments, the present disclosure provides a method of decreasing serum biomarker levels associated with the development of CRS or an associated disorder in a patient, the method comprising administering a therapeutically effective amount of a chimeric amphotropic RNA vector (e.g., CAR-V™ vector) encoding an anti-cyclin G1 construct or two killer genes (anti-cyclin G1 and HSV-tk) to the patient. In some embodiments, the CAR-V™ vector is administered to the patient until the serum biomarker levels decrease to levels observed in patients who do not develop CRS. In some embodiments, the CAR-V™ vector is administered to the patient until the serum biomarker levels decrease to levels observed in the same patient before immunotherapy treatment. In some embodiments, the biomarker comprises at least one of IL-6, IL-6R, C-reactive protein (CRP), IL-10, sIL-2R, TNF-α, lactate dehydrogenase (LDH), ferritin, G-CSF, sodium, creatinine, CD25, or CD69.

In some embodiments, the present disclosure provides a method of preventing, treating, or lessening the effects of CRS or an associated disorder in a patient, the method comprising a step of determining the expression of a biomarker associated with CRS or an associated disorder in the patient and a step of administering a therapeutically effective amount of a chimeric amphotropic RNA vector (e.g., CAR-V™ vector) encoding an anti-cyclin G1 construct or two killer genes (anti-cyclin G1 and HSV-tk) to the patient. In these embodiments, the step of determining the expression of the biomarker associated with CRS or an associated disorder may be performed prior to administering the chimeric amphotropic RNA vector (e.g., CAR-V™ vector). The step of determining the expression of the biomarker may be performed prior to the administration of the treatment in order to assess baseline levels and guide the selection of an appropriate therapeutic approach.

In some embodiments, the present disclosure provides a method of preventing, treating, or lessening the effects of CRS or an associated disorder in a patient, the method comprising a step of administering a therapeutically effective amount of a chimeric amphotropic RNA vector (e.g., CAR-V™ vector) encoding an anti-cyclin G1 construct or two killer genes (anti-cyclin G1 and HSV-tk) to the patient and a step of determining the expression of a biomarker associated with CRS or an associated disorder in the patient. In these embodiments, the step of determining the expression of the biomarker associated with CRS or an associated disorder may be performed after administering the chimeric amphotropic RNA vector (e.g., CAR-V™ vector).

The step of determining the expression of the biomarker may be performed after the administration of the treatment in order to evaluate the therapeutic response and adjust the treatment protocol as necessary.

In some embodiments, the present disclosure provides a method of preventing, treating or lessening the effects of cytokine release syndrome (CRS) or an associated disorder in a patient, the method comprising administering a therapeutically effective amount of a chimeric amphotropic RNA vector (e.g., CAR-V™ vector) encoding an anti-cyclin G1 construct or two killer genes (anti-cyclin G1 and HSV-tk) to the patient, wherein the chimeric amphotropic RNA vector (e.g., CAR-V™ vector) is administered at a dose of at least about 0.6×10¹⁰ RV copies or at a dose of from about 0.6×10¹⁰ RV to about 6×10¹⁰ RV copies.

In some embodiments, the present disclosure provides a method of preventing, treating or lessening the effects of cytokine release syndrome (CRS) or an associated disorder in a patient, the method comprising administering a therapeutically effective amount of a chimeric amphotropic RNA vector (e.g., CAR-V™ vector) encoding an anti-cyclin G1 construct or two killer genes (anti-cyclin G1 and HSV-tk) to the patient, wherein, in particular administering a dose of the chimeric amphotropic RNA vector (e.g., CAR-V™ vector) to the subject daily for a period of from about 3 days to about 14 days, or from about 7 days to about 28 days.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an illustration of an embodiment of the present disclosure, more specifically the mechanism of action of chimeric amphotropic RNA vectors (e.g., CAR-V™ vectors, DeltaRex-G or DNG64-CAR-V™, DeltaRex-GT or DNG79-CAR-V™), in CAR-T cell induced severe CRS.

### DETAILED DESCRIPTION OF THE INVENTION

The term “about” may be used in conjunction with numerical values and/or ranges.

The term “about” is understood to mean those values near to a recited value. For example, “about 0.6×10¹⁰ RV copies” may mean within ±10% of 0.6×10¹⁰ RV copies, within ±10%, ±9%, ±8%, ±7%, ±7%, +5%, +4%, +3%, +2%, ±1%, less than ±1%, or any other value or range of values therein.

Throughout the present specification, numerical ranges comprise all subranges therein. For example, the range “from 20 to 150” includes all possible ranges therein (e.g., 21-149, 30-140, 50-100, etc.). Additionally, all values within a given range may be an endpoint for the range encompassed thereby (e.g., the range 20-150 includes the ranges with endpoints such as 20-100, 90-150, etc.).

Subjects to be treated by the methods of the disclosed embodiments are human subjects and animal subjects (e.g., horse, dog, cat, monkey, chimpanzee, and/or any mammalian species or the like) for veterinary purposes. The subjects may be male or female and may be any suitable age, e.g., neonatal, infant, juvenile, adolescent, adult, or geriatric. In some embodiments, the subjects are of the mammalian species.

The present disclosure provides methods of preventing, lessening the effects, or treating Cytokine Release Syndrome (CRS) or an associated disorder in a patient, comprising administering a therapeutically effective amount of a chimeric amphotropic RNA vector (e.g., CAR-V™ vector) encoding an anti-cyclin G1 construct or two killer genes (an anti-cyclin G1/HSV-tk) to the patient, wherein the patient has received, is receiving or about to receive an immunotherapy.

As used herein, the term “chimeric amphotropic RNA vector encoding an anti-cyclin G1 construct or two killer genes (an anti-cyclin G1/HSV-tk)” or “CAR-V™ vector encoding an anti-cyclin G1 construct or two killer genes (an anti-cyclin G1/HSV-tk)” refers to a chimeric amphotropic RNA vector displaying a SIG-binding decapeptide (i.e., the vector surface membrane envelope (env) protein, gp70, has been modified by inserting a SIG-binding decapeptide in the gp70 env protein by molecular engineering) encoding a cytocidal anti-cyclin G DNA construct or two killer genes (anti-cyclin G1 and HSV-tk). CAR-V™ displays a SIG binding peptide that hunts down Signature (SIG) proteins in damaged tissues and delivers a cytocidal gene or genes of interest which after random integration in the nucleus of dividing cells (e.g. immune cells) express an anti-cyclin G1 and Herpes Simplex Virus thymidine kinase (HSVtk) protein, antibody, or RNA molecule. It specifically targets cyclin G1, a regulatory protein involved in the control of the cell cycle and cell cycle checkpoints, or the HSVtk protein, which induce cell death (e.g. immune cells, cancer cells) upon administration of a prodrug (e.g. valacyclovir). The anti-cyclin G1 construct is designed to modulate or inhibit the function of cyclin G1 and valacyclovir prodrug is designed to phosphorylate thymidine kinase which is toxic to cells.

Other uses of chimeric amphotropic RNA vectors (e.g., CAR-V™ vectors), methods for their production and testing are described in the following patents and patent applications, each of which is hereby incorporated by reference in its entirety: U.S. Pat. No. 11,325,958 B2, U.S. Pat. No. 12,173,041 B2, US 2021/0299276 A1, US 2021/0346520 A1, US 2023/0080185 A1.

The patient who receives the chimeric amphotropic RNA vector (e.g., CAR-V™ vector) treatment under the methods of the present disclosures, has received, is receiving or about to receive an immunotherapy, wherein the immunotherapy is a Chimeric Antigen Receptor T cell (CAR-T) therapy, an antibody-based therapy, a Natural Killer cell (NK-cell) therapy, an Immune Checkpoint Inhibitors (ICI) therapy, a Tumor-Infiltrating Lymphocytes (TIL) therapy, cytokine therapy, Bispecific T-cell Engagers (BiTEs) therapy, a neoTCR-T or any other Adoptive Cell Transfer (ACT) therapy.

As used herein, the term “chimeric antigen receptor (CAR) therapy” refers to an immunotherapy in which T cells are genetically modified to express a synthetic receptor, called a chimeric antigen receptor (CAR). The fusion protein comprises an extracellular domain capable of specifically binding to an antigen, a transmembrane domain sourced from a different polypeptide than the extracellular domain, and at least one intracellular domain. This construct is also known as a “chimeric receptor”, “T-body” or “chimeric immune receptor (CIR)” facilitates targeted recognition and signaling in immune cells. The term “extracellular domain capable of binding to an antigen” encompasses any oligopeptide or polypeptide with antigen-binding capability. Similarly, the term “intracellular domain” denotes any oligopeptide or polypeptide recognized for its role in transmitting signals that modulate biological processes within a cell. The modified T-cells recognize and bind to antigens present on the surface of target cells, such as tumor cells. This modification enhances the ability of T cells to identify and eliminate cells expressing the target antigen, thus providing a targeted therapeutic approach for various cancers and other diseases.

The term “Natural Killer cell (NK-cell) therapy” refers to an immunotherapy in which Natural Killer (NK) cells are utilized to recognize and destroy infected or tumorigenic cells without prior sensitization. In NK-cell therapy, these cells are either isolated from the patient or sourced from a donor, and may be expanded, activated, or genetically modified before being reintroduced into the patient to enhance their ability to target and eliminate cancerous or infected cells. NK cells may also bear Chimeric Antigen Receptors (CARs) known as CAR-NK cell therapy.

The term “Immune Checkpoint Inhibitors (ICI) therapy” refers to an immunotherapy based on pharmaceutical agents designed to inhibit immune checkpoint proteins, such as PD-1, PD-L1, or CTLA-4. These proteins found in regulatory T cells or tumor cells normally function to suppress the immune system's ability to detect and eradicate tumor cells by inhibiting T cell activation and function. By disrupting these inhibitory signals, ICI therapy restores and enhances the ability of the immune system to recognize and eliminate neoplastic cells, thereby augmenting the anti-tumor immune response. Immune checkpoint inhibitors, such as ipilimumab (YERVOY®) and nivolumab (OPDIVO®), have demonstrated substantial efficacy in the treatment of various malignancies, including melanoma, non-small cell lung carcinoma, and renal cell carcinoma.

The term “Tumor-Infiltrating Lymphocytes (TIL) therapy” refers to an immunotherapy in which autologous T lymphocytes that have naturally infiltrated a tumor are isolated, expanded and reintroduced into the patient's body to enhance the immune system's ability to recognize and eliminate cancer cells. This therapy leverages the tumor-specific cytotoxic activity of TILs. TIL therapy has shown promising results, particularly in treating cancers such as melanoma and other solid tumors.

The term “cytokine therapy” refers to an immunotherapy based on cytokines to enhance the immune response against cancer or infections. Cytokines, including interleukins (e.g., IL-2, IL-12, IL-15) and interferons (e.g., IFN-α) are administered exogenously to stimulate or modulate the activity of immune cells, including T cells, natural killer (NK) cells, macrophages and dendritic cells.

The term “Bispecific T-cell Engagers (BiTEs) therapy” refers to an immunotherapy based on bispecific monoclonal antibodies designed to bind simultaneously to both T cells and tumor cells. These antibodies have two binding sites-one targeting a specific tumor-associated antigen on cancer cells and another that binds to CD3, a protein on T cells. By bridging the T cells and tumor cells, BiTEs promote the activation of T cells, leading to the targeted destruction of tumor cells.

The term “Adoptive Cell Transfer (ACT) therapy” refers to an immunotherapy in which immune cells, including T cells, are collected from a patient, expanded or genetically modified ex vivo, and then reintroduced into the patient's body to enhance anti-tumor immunity. ACT therapies include approaches such as Chimeric Antigen Receptor (CAR) T-cell therapy, TIL and neoT Cell Receptor-T cell (neoTCR-T) therapy.

These therapies may have side effects, including Cytokine Release Syndrome (CRS). CRS can vary in severity, from mild symptoms to life-threatening conditions requiring intervention.

Cytokine release syndrome (CRS) is a form of systemic inflammatory response syndrome (SIRS) that can arise as a complication of some diseases or infections. It may be an adverse effect of certain immunotherapies described herein, including a Chimeric Antigen Receptor T cell (CAR-T) therapy, an antibody-based therapy, a Natural Killer cell (NK-cell) therapy, an Immune Checkpoint Inhibitors (ICI) therapy, a Tumor-Infiltrating Lymphocytes (TIL) therapy, a cytokine therapy, a Bispecific T-cell Engagers (BiTEs) therapy, a neoTCR-T therapy or any other Adoptive Cell Transfer (ACT) therapy.

The term “associated disorder in a patient” in the context of CRS refers to a pathological condition that results from CRS, including but not limited to organ damage (e.g., cardiovascular, respiratory, integumentary, gastrointestinal, hepatic, renal, hematological, and nervous systems), capillary leak syndrome, coagulopathy, neurotoxicity, hypotension, and multi-organ dysfunction.

CRS can impact multiple organ systems, including the cardiovascular, respiratory, integumentary, gastrointestinal, hepatic, renal, hematological, and nervous systems. Patients at heightened risk of experiencing severe CRS often include individuals with extensive disease burden, pre-existing co-morbidities, or those who exhibit early onset CRS within the first three days following the immunotherapy cell infusion (e.g., CAR-T). Elevated serum levels of specific biomarkers, including IL-6, IL-6R, C-reactive protein (CRP), IL-10, sIL-2R, TNF-α, lactate dehydrogenase (LDH), ferritin, G-CSF, sodium, creatinine, CD25, or CD69, measured either before or within 24 hours of cell infusion, are correlated with an increased likelihood of severe CRS development. Typically, the intensity of the inflammatory response is influenced by a dynamic balance between proinflammatory and anti-inflammatory mechanisms, which helps maintain immune homeostasis. This balance is finely regulated through complex networks involving various immune cells, including lymphocytes (B cells, T cells, and natural killer cells), myeloid cells (macrophages, dendritic cells, and monocytes), and endothelial cells. Furthermore, each cytokine can have both stimulating and suppressing effects on other cytokines, contributing to a cytokine network that governs this regulatory balance. In some embodiments, the associated disorder is or comprises organ damage resulting from the pathological effects of CRS.

In some embodiments, the associated disorder is or comprises organ damage affecting one or more organs selected from the group consisting of lung, kidney, liver, heart, and brain.

An exemplary method of the present disclosure comprises administering a therapeutically effective amount of a chimeric amphotropic RNA vector (e.g., CAR-V™ vector) encoding an anti-cyclin G1 construct or two killer genes (an anti-cyclin G1/HSV-tk) to the patient, wherein the patient has received, is receiving or about to receive an immunotherapy.

The chimeric amphotropic RNA vectors (e.g., CAR-V™ vectors) encoding an anti-cyclin G1 construct or two killer genes (an anti-cyclin G1/HSV-tk) not only modulate the cancer cell activity, but also the immune cell activity, reducing the severity of CRS without eliminating the efficacy of immunotherapy.

In the case of an immunotherapy based on cell-based therapy (such as CAR-T therapy), unlike steroids, the chimeric amphotropic RNA vectors (e.g., CAR-V™ vectors) selectively target the cells that are actively dividing at a given time at sites of tissue injury, where sequences of the Signature (SIG) proteins are exposed. As described in U.S. Pat. No. 12,173,041 B2, the Sig sequence binding peptide binds a Gly-Xxx-Pro/Hyp-Ala-Xxx-Pro/Hyp, wherein Xxx is an amino acid other than Gly, Pro, or Hyp (SEQ ID NO: 3). Thus, the cells that are not actively dividing at the time of treatment are preserved. This is one of the advantages of the method of the present disclosures which effectively prevent, treat or lessen the symptoms of CRS or an associated disorder, without impacting the efficacy of the associated immunotherapy.

In some embodiments, the method of the present disclosure comprises:
a) administering a therapeutically effective amount of an immunotherapy to a patient;
b) administering a therapeutically effective amount of a chimeric amphotropic RNA vector (e.g., CAR-V™ vector) encoding an anti-cyclin G1 construct or two killer genes (an anti-cyclin G1/HSV-tk) to a patient;
wherein step a) takes place prior to step b), simultaneously with step b) or after step b); and
wherein steps a) and/or b) are optionally repeated several times.

In some embodiments, the method of the present disclosure comprises in this order:
a) administering a therapeutically effective amount of an immunotherapy to a patient; and
b) administering a therapeutically effective amount of a chimeric amphotropic RNA vector (e.g., CAR-V™ vector) encoding an anti-cyclin G1 construct or two killer genes (an anti-cyclin G1/HSV-tk) to a patient.

In some embodiments, the therapeutically effective amount of a chimeric amphotropic RNA vector (e.g., CAR-V™ vector) encoding an anti-cyclin G1 construct or two killer genes (an anti-cyclin G1/HSV-tk) is administered to the patient after the immunotherapy, when the immunotherapy is effective, i.e., when the immunotherapy is functional, active, engrafted (for cell-based therapies including CAR-T therapy), eliciting a therapeutic response, or achieving immune activation.

The immunotherapy can be administered in several doses, depending on the specific treatment protocol, the type of immunotherapy being used, and the patient's condition. The dosing schedule may vary and is typically designed to maximize therapeutic efficacy while minimizing side effects.

The timing and frequency of the chimeric amphotropic RNA vectors (e.g., CAR-V™ vectors) administration are typically tailored to the patient's response, aiming to optimize the therapeutic outcome while minimizing CRS-related complications.

Dosing prior to and after the development of CRS or the onset of toxicity may help determine the optimal timing and dosage needed to control or mitigate the effects of CRS, thereby guiding the establishment of the most effective therapeutic protocol tailored to the individual patient's needs.

In some embodiments, the method of the present disclosure comprises:
a) determining the expression of a biomarker associated with CRS in a patient prior to administering a therapeutically effective amount of a chimeric amphotropic RNA vector (e.g., CAR-V™ vector) encoding an anti-cyclin G1 construct or two killer genes (an anti-cyclin G1/HSV-tk); and
b) administering a therapeutically effective amount of the chimeric amphotropic RNA vector (e.g., CAR-V™ vector) encoding an anti-cyclin G1 construct or two killer genes (an anti-cyclin G1/HSV-tk) to the patient.

In some embodiments, the method of the present disclosure comprises:
a) administering a therapeutically effective amount of a chimeric amphotropic RNA vector (e.g., CAR-V™ vector) encoding an anti-cyclin G1 construct or two killer genes (an anti-cyclin G1/HSV-tk) to a patient; and
b) determining the expression of a biomarker associated with CRS in the patient after administering the therapeutically effective amount of the chimeric amphotropic RNA vector (e.g., CAR-V™ vector) encoding an anti-cyclin G1 construct or two killer genes (an anti-cyclin G1/HSV-tk).

In some embodiments, the method of the present disclosure comprises:
a) determining the expression of a biomarker associated with CRS in a patient prior to administering a therapeutically effective amount of a chimeric amphotropic RNA vector (e.g., CAR-V™ vector) encoding an anti-cyclin G1 construct or two killer genes (an anti-cyclin G1/HSV-tk);
b) administering the therapeutically effective amount of a chimeric amphotropic RNA vector (e.g., CAR-V™ vector) encoding an anti-cyclin G1 construct or two killer genes (an anti-cyclin G1/HSV-tk) to the patient; and
c) determining the expression of a biomarker associated with CRS in the patient;
d) optionally repeating steps b) and c) as needed until the biomarker level decreases to levels observed prior to the immunotherapy or to baseline levels observed in the same patient.

The chimeric amphotropic RNA vector (e.g., CAR-V™ vector) may be administered to the patient before the development of CRS, or symptoms thereof, and/or after the development of CRS, or symptoms thereof. For example, the CAR-V™ vector may be administered to the patient before the onset of the CRS toxicity, or symptoms thereof, and/or after the onset of the CRS toxicity, or symptoms thereof. In some embodiments, the chimeric amphotropic RNA vector (e.g., CAR-V™ vector) is administered to the patient in multiple doses, both prior to and after the development of CRS, for example before and after the onset of the CRS toxicity, or symptoms thereof.

The onset of CRS toxicity may occur within the first weeks after immunotherapy (e.g., CAR-T cell therapy or monoclonal antibody) administration, for example within the first weeks after immunotherapy, for example after the first dose of immunotherapy. For example, CRS toxicity may peak within 1 to 2 weeks of administration of the immunotherapy. The onset of CRS cytotoxicity may be predicted. It is typically determined through clinical monitoring, laboratory tests, and assessment of symptoms. The onset of CRS toxicity may notably be monitored by assessing the levels of specific biomarkers, such as IL-6 or TNF-α, in the serum of the patient. Additionally, clinical symptoms such as fever, hypotension, organ dysfunction, and respiratory distress may be closely observed to detect early signs of CRS.

In some embodiments, the method of the present disclosure may comprise administering a therapeutically effective amount of a chimeric amphotropic RNA vector (e.g., CAR-V™ vector) encoding an anti-cyclin G1 construct or two killer genes (an anti-cyclin G1/HSV-tk) to the patient prior to the onset of CRS toxicity. In these embodiments, the administration of chimeric amphotropic RNA vector (e.g., CAR-V™ vector) may occur prior to the patient receiving the immunotherapy, at substantially the same time as the patient is receiving the immunotherapy or following the administration of the immunotherapy.

In some embodiments, the method of the present disclosure may comprise administering a therapeutically effective amount of a chimeric amphotropic RNA vector (e.g., CAR-V™ vector) encoding an anti-cyclin G1 construct or two killer genes (an anti-cyclin G1/HSV-tk) to the patient after the onset of CRS toxicity. In these embodiments, the administration of CAR-V vector occurs following the administration of the immunotherapy.

In some embodiments, the method of the present disclosure may comprise administering several therapeutically effective amounts of a chimeric amphotropic RNA vector (e.g., CAR-V™ vector) encoding an anti-cyclin G1 construct or two killer genes (an anti-cyclin G1/HSV-tk) to the patient prior to the onset of CRS toxicity, for example several doses of CAR-V vector prior to the onset of CRS toxicity.

In some embodiments, the method of the present disclosure may comprise administering several therapeutically effective amounts of a chimeric amphotropic RNA vector (e.g., CAR-V™ vector) encoding an anti-cyclin G1 construct or two killer genes (an anti-cyclin G1/HSV-tk) to the patient after the onset of CRS toxicity, for example several doses of CAR-V vector prior to the onset of CRS toxicity.

In some embodiments, the method of the present disclosure may comprise administering several therapeutically effective amounts of a chimeric amphotropic RNA vector (e.g., CAR-V™ vector) encoding an anti-cyclin G1 construct or two killer genes (an anti-cyclin G1/HSV-tk) to the patient prior and after the onset of CRS toxicity. For example, the patient may receive one or several doses of a chimeric amphotropic RNA vector (e.g., CAR-V™ vector) prior to the onset of CRS toxicity, as well as one or several doses of a chimeric amphotropic RNA vector (e.g., CAR-V™ vector) after the onset of CRS toxicity.

In some embodiments, the method of the present disclosure comprises:
a) administering a therapeutically effective amount of an immunotherapy to a patient;
b) administering a therapeutically effective amount of a chimeric amphotropic RNA vector (e.g., CAR-V™ vector) encoding an anti-cyclin G1 construct or two killer genes (an anti-cyclin G1/HSV-tk) to the patient;
c) determining the expression of a biomarker associated with CRS in the patient;
d) optionally repeating steps b) and c) as needed until the biomarker level decreases to levels observed prior to the immunotherapy or to baseline levels observed in the same patient; wherein step a) may take place prior to step b), simultaneously with step b) or after step b); and wherein step c) may take place prior to step b) and/or after step b); and Steroids, including corticosteroids, may be used to suppress inflammatory responses and are sometimes indicated in the management of CRS associated with immunotherapies. The method of the present disclosure may comprise a step of administering a steroid therapy, in particular administering a therapeutically effective amount of a steroid therapy.

In some embodiments, the method of the present disclosure comprises in this order:
a) administering a therapeutically effective amount of an immunotherapy to a patient;
b) administering a therapeutically effective amount of a chimeric amphotropic RNA vector (e.g., CAR-V™ vector) encoding an anti-cyclin G1 construct or two killer genes (an anti-cyclin G1/HSV-tk) to a patient; and
c) administering a therapeutically effective amount of a steroid to a patient;
wherein:
steps a), b) and c) may be carried out in any order;
step a) is carried out before steps b) and c);
step a) is carried out before step b), and step b) is carried out before step c); or
step a) is carried out before step c), and step c) is carried out before step b).

The steroid therapy may be administered to the patient before the administration of the immunotherapy, at the same time as the administration of the immunotherapy, and/or after the administration of the immunotherapy. The steroid therapy may be administered to the patient before the administration of the chimeric amphotropic RNA vectors (e.g., CAR-V™ vectors) disclosed herein, at the same time as the administration of the chimeric amphotropic RNA vectors (e.g., CAR-V™ vectors), and/or after the administration of the chimeric amphotropic RNA vectors (e.g., CAR-V™ vectors).

In some embodiments, the steroid therapy is administered to the patient after the administration of the immunotherapy. For example, the steroid therapy may be administered to the patient less than about 3 hours after the administration of the immunotherapy, less than about 2 hours, or less than about 1 hour after the administration of the immunotherapy.

The steroid therapy may be administered to the patient before the onset of the CRS toxicity, or symptoms thereof, and/or after the onset of the CRS toxicity, or symptoms thereof.

In some embodiments, the steroid therapy is administered to the patient after the onset of the CRS toxicity, or symptoms thereof. For example, the steroid therapy may be administered to the patient less than about 3 hours after the onset of the CRS toxicity, less than about 2 hours, or less than about 1 hour after the onset of the CRS toxicity.

In some embodiments, the steroid therapy is administered to the patient in multiple doses, both prior to and after the development of CRS, for example before and after the onset of the CRS toxicity, or symptoms thereof.

In some embodiments, the steroid therapy is administered to the patient in multiple doses, after the development of CRS, for example after the onset of the CRS toxicity, or symptoms thereof.

The steroid therapy may be administered to the patient before the administration of the chimeric amphotropic RNA vectors (e.g., CAR-V™ vectors) disclosed herein, at the same time as the administration of the CAR-V™ vectors, and/or after the administration of the chimeric amphotropic RNA vectors (e.g., CAR-V™ vectors).

While steroids are sometimes indicated in the management of CRS associated with immunotherapies as they suppress inflammatory responses, they can also impair T-cell function and/or induce T-cell apoptosis, compromising the efficacy of immunotherapy. In some embodiments, the method of the present disclosure does not rely on steroids, including corticosteroids, for CRS management, thereby preserving the therapeutic activity of the administered immunotherapy.

In some embodiments, the immunotherapy is a CAR-T cells therapy. In these embodiments, the chimeric amphotropic RNA vectors (e.g., CAR-V™ vectors) may be administered to the patient when the CAR-T cells are engrafted, i.e., when they have survived, proliferated and established themselves in the patient's body. As such, the CAR-T cells population is established and preserved following the administration of chimeric amphotropic RNA vectors (e.g., CAR-V™ vectors). Indeed, unlike steroids, the chimeric amphotropic RNA vectors (e.g., CAR-V™ vectors) only target the CAR-T cells that are actively dividing at a given time at the site of injured tissues where collagenous SIG proteins are exposed. Thus, the CAR-T cells that are not actively dividing at the time of treatment are preserved.

An exemplary method of the present disclosure may comprise a step of determining the levels of CAR-T cells circulating in the patient's blood stream after administering a therapeutically effective amount of the chimeric amphotropic RNA vector (e.g., CAR-V™ vector) or upon completion of the treatment protocol.

The chimeric amphotropic RNA vectors (e.g., CAR-V™ vectors) utilized in the methods of the present invention are non-immunogenic. The vectors do not elicit an immune response in the patients, thereby ensuing their efficacy in targeted therapeutic applications. The vectors are engineered with a specific peptide sequence, a decapeptide, that selectively binds to aberrant proteins present in the tumor microenvironment (TME). Upon binding, the vectors utilize a receptor on the surface of target cells, facilitating cellular entry and subsequent cytotoxicity. The vectors specifically target highly proliferative cells, including malignant tumor cells, neoangiogenic endothelial cells, and stroma-producing fibroblasts, while sparing normal, non-proliferative cells.

The chimeric amphotropic RNA vector (e.g., CAR-V™ vector) formulation may be administered to the patient parenterally, including intravenously. In some embodiments, the vector formulation is administered to the patient via intravenous infusion at a controlled rate. The controlled rate may be about 3 mL to 6 mL per minute, for example about 4 mL per minute.

The chimeric amphotropic RNA vector (e.g., CAR-V™ vector) may be administered to the patient as a single dose or in multiple doses over a defined period, depending on the therapeutic regimen or the levels of certain biomarkers described herein.

The formulation is typically an aqueous solution ensuring a rapid and complete bioavailability. It may include buffering agents, stabilizers and tonicity adjusters to maintain stability, enhance delivery, and optimize physiological compatibility.

In some embodiments, the formulation comprises between about 0.6×10¹⁰ RV copies or at a dose of from about 0.6×10¹⁰ RV copies to about 6×10¹⁰ RV copies of the chimeric amphotropic RNA vector (e.g., CAR-V™ vector).

The formulation may comprise one or several buffering agents, such as phosphate, citrate, acetate, or tris(hydroxymethyl)aminomethane (Tris).

The formulation may comprise one or several excipients, including preservatives, salts, pH adjusting agents.

The term “excipient,” as used herein, refers to any substance that may be formulated with the chimeric amphotropic RNA vector (e.g., CAR-V™ vector) and may be included for the purpose of enhancement of the chimeric amphotropic RNA vector (e.g., CAR-V™ vector) in the final dosage form, such as facilitating its bioavailability, reducing viscosity and/or osmolality, enhancing solubility of the composition or to enhance long-term stability. Excipients can also be useful in the manufacturing process, to aid in the handling of the active substance. The selection of appropriate excipients also depends upon the route of administration and the dosage form, as well as the active ingredient and other factors. Accordingly, the chimeric amphotropic RNA vector (e.g., CAR-V™ vector) may be combined with any excipient(s) known in the art that allows tailoring its performance during manufacturing or administration as well as its in vitro and in vivo performance. Many of these excipients may be utilized to tailor the pharmacokinetic profiles of chimeric amphotropic RNA vector (e.g., CAR-V™ vector) formulations.

The term “buffer” or “buffering agent,” as used herein, refers to a solution which resists changes in the hydrogen ion concentration on the addition of a small amount of acid or base. This includes, for example, a weak acid or base that is used to maintain the pH of a solution near a chosen pH value after the addition of another acidic or basic compound. The function of such buffer or buffering agent is to prevent a change in pH of a solution when acids or bases are added to said solution.

The term “pH adjusting agent,” as used herein, refers to an acid or base used to alter the pH of a solution to a chosen pH value. The function of such an agent is to alter the pH of a solution to the desired value subsequent to the addition of acidic or basic compounds.

The term “formulation,” as used herein, refers to compositions for therapeutic use, including, for example, a stable and pharmaceutically acceptable preparation of a pharmaceutical composition or formulation disclosed herein.

The term “aqueous formulation,” as used herein, refers to a water-based formulation, in particular, a formulation that is an aqueous solution.

The term “high concentration chimeric amphotropic RNA vector formulations” as used herein, refers to those formulations where the chimeric amphotropic RNA vector (e.g., CAR-V™ vector) concentration is about 6×10⁸ retroviral vector (RV) copies or higher, or about 6×10⁸ RV copies or higher.

The term “pharmacokinetic” or “PK” as used herein, refers to in vivo movement of an individual agent in the body, including the plasma concentration time profiles and kinetic parameters like the maximum concentration (Cmax), area under the curve (AUC), and time to maximum concentration of said agent (Tmax).

The phrase “pharmaceutically acceptable” or “acceptable”, as used in connection with compositions of the disclosure, refers to molecular entities and other ingredients of such compositions that are physiologically tolerable and do not typically produce untoward reactions when administered to an animal and/or human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “physiologically relevant” as used herein, refers to a measurement, level or amount that is suitable for use in a pharmaceutical, therapeutic or other dosage form to be administered to an animal subject, particularly a human subject.

As used herein, the term “parenteral” refers to any non-oral means of administration. It includes intravenous (i.v. or IV) infusion, IV bolus injection, subcutaneous (s.c. or SC) and intramuscular (i.m. or IM) injection.

The chimeric amphotropic RNA vector (e.g., CAR-V™ vector) formulation may be stored in sealed containers, such as vials or cryobags. It may be stored as temperature below −70° C. until use, for example at a temperature between −75° C. and −90° C. Prior to administration, the formulation may be thawed by placing the container in a water bath maintained at a temperature above 30° C., for example between 32° C. and 37° C., for a time period of at least 10 minutes, for example between 15 to 40 minutes.

In some embodiments, the chimeric amphotropic RNA vector (e.g., CAR-V™ vector) is based on a non-replicating vector, ensuring controlled gene delivery without viral propagation in the host. The vector may be retroviral or non-retroviral.

In some embodiments, the chimeric amphotropic RNA vector (e.g., CAR-V™ vector) is based on murine leukemia virus (MLV), Moloney murine leukemia virus (MoMLV), leukemogenic murine leukemia virus (LMLV), or other retroviral vectors such as lentiviral vectors (LV, e.g., HIV-1-based, SIV-based, FIV-based), alpharetroviral vectors (ARV, e.g., ALV-based), or foamy virus vectors (FV, e.g., Spumaviruses), as well as non-retroviral options such as adeno-associated virus (AAV) and adenoviral vectors, each with distinct integration and expression properties.

In some embodiments, the chimeric amphotropic RNA vector (e.g., DNG64-CAR-V™ vector) is based on murine leukemia virus (MLV) and is designed to target abnormal Signature (Sig) proteins within the tumor microenvironment (TME), enabling selective delivery of therapeutic genes to malignant tissues. As described in U.S. Pat. No. 12,173,041 B2, the Sig sequence binding peptide binds a Gly-Xxx-Pro/Hyp Ala-Xxx-Pro/Hyp, wherein Xxx is an amino acid other than Gly, Pro, or Hyp (SEQ ID NO: 3). It achieves this by displaying a SIG binding peptide on its gp70 surface membrane, and encoding a dominant negative mutant construct of human cyclin G1. The vector comprises a neomycin resistance (neo^{r}) gene that is controlled by the SV40 early promoter.

In some embodiments, the chimeric amphotropic RNA vector (e.g., CAR-V™ vector) is based at least one of DNG64 or DNG79. These vectors may be produced by transiently co-transfecting human embryonic kidney 293T-cells (HEK293T) with 3 proprietary plasmid DNAs.

An exemplary method of the present disclosure may comprise one or several additional steps, including a step of detecting the presence of anti-vector antibodies in the patient's serum, a step of testing for the presence of replication-competent retroviruses (RCRs), and/or a step of performing vector DNA integration studies in the patient's peripheral blood lymphocytes.

Mechanism of action of chimeric amphotropic RNA vectors (e.g., CAR-V™ vectors) encoding an anti-cyclin G1 construct (DNG64-CAR-V™) or two killer genes (an anti-cyclin G1/HSV-tk gene, DNG79-CAR-V™).

The chimeric amphotropic RNA vectors of the present disclosure are vectors encoding an anti-cyclin G1 construct (DNG64-CAR-V™) or two killer genes (an anti-cyclin G1/HSV-tk gene; DNG79-CAR-V™). They typically are tumor targeted retrovectors encoding a cytocidal CCNG1 gene which inhibits cyclin G1 expression, and consequently, prohibit cell cycle progression.

In some embodiments, the chimeric amphotropic RNA vector utilized in the method of the present disclosure is at least one of DNG64-CAR-V™ or DNG79-CAR-VTM These vectors are equipped with membrane gp70 envelopes that incorporate a signature (SIG) protein-binding decapeptide. This decapeptide recognizes and binds to abnormal anaplastic SIG proteins in the tumor microenvironment (TME). Upon binding, the vectors enter the target cells via the innate amphotropic Pit2 receptor.

These vectors encode a dominant negative mutant construct of the cyclin G1 (CCNG1) gene that is devoid of its N-terminal domain and the first two helical segments (α1 and α2) of the definitive cyclin, thereby impairing its proteolytic processing. Upon cellular entry, the vector-derived RNA is reverse-transcribed into DNA, which subsequently translocates to the nucleus of actively dividing cancer cells. The integrated transgene is incorporated into the host genome, leading to sustained expression of the cytocidal CCNG1 inhibitor. This enforced expression disrupts cell cycle progression by arresting cells in the G1 phase, ultimately inducing apoptosis in in highly proliferative cells, including cancer cells, neoangiogenic cells and stroma-producing fibroblasts, while sparing healthy tissues.

The DNG64-CAR-V™ or DNG79-CAR-V™ vectors can invade rapidly proliferating tumor cells to integrate its viral genome and produce cytocidal cyclin G1, which inhibits cell cycle progression at the G0-G1 phase and triggers cell death via apoptosis.

Furthermore, these vectors are efficient in inhibiting cell cycle progression in cytokine-producing T-cells and activated macrophages. Death of cytokine-producing T-cells and activated macrophages reduce the severity of CRS. Through the same mechanism employed in cancer cells, the vector inhibits cell cycle progression at the GO-G1 phase and induces apoptosis in cytokine-producing T cells and activated macrophages. This targeted cytotoxic effect helps suppress excessive immune activation, thereby mitigating inflammatory responses such as those observed in CRS.

The efficacy of these vectors in mitigating overstimulated immune response has been demonstrated in the context of Acute Respiratory Distress Syndrome (ARDS), a condition associated with mortality and morbidity in COVID-19 patients. During the Covid-19 pandemic, DeltaRex-G was granted FDA Emergency Use Authorization for severe Covid-19-induced CRS and acute respiratory distress syndrome (ARDS). CRS and ARDS develop from excessive stimulation of activated immune cells and consequent cytokine release, provoking further immune cell attack and tissue damage.

Importantly, the DNG64-CAR-V™ or DNG79-CAR-V™ vectors are not immunogenic hence preventing CRS or ARDS flare-ups.

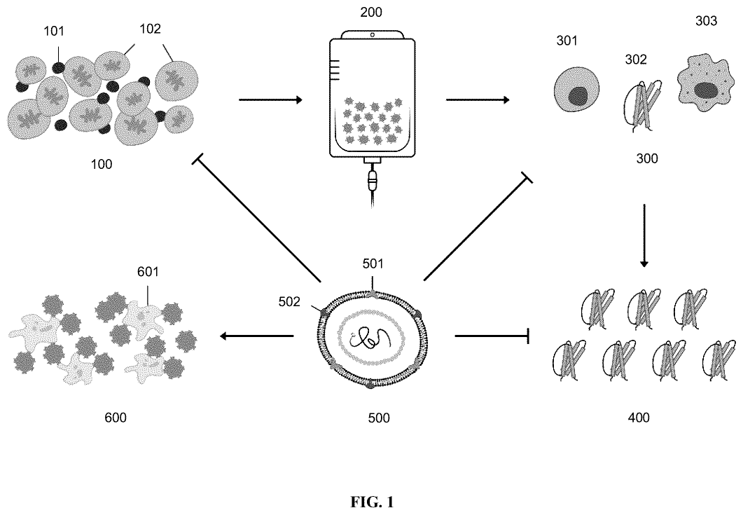

FIG. 1 illustrates the progression of immune activation within a high tumor burden microenvironment 100 (TME) characterized by inflammation. Initially, the TME exhibits a dense population of tumor cells, including mitotic cancer cells 102 actively proliferating, along with tumor-infiltrating lymphocytes 101 (TILs) attempting to mount an immune response. Chimeric Antigen Receptors (CARs) T cells 200 are engineered to recognize and bind antigens specific to the patient's cancer cells and are subsequently inserted into T cells harvested from the patient. Upon administration of CAR-T cells to the patient, the immune activation subsequently triggers the recruitment and activation of monocytes 301 and macrophages 303, leading to an amplified inflammatory response 300. As a result, there is a pronounced release of pro-inflammatory cytokines, particularly interleukin-6 302 (IL-6 ), which is a hallmark of cytokine release syndrome 400 (CRS) and contributes to systemic immune dysregulation. Patients with high IL-6 levels and large baseline tumor burdens have inflammatory TMEs that prime myeloid cells and macrophages to induce an immune response, and this condition is amplified by CAR-T cell treatment. Elevated IL-6 levels post-treatment have been correlated with diminished response to CAR-T cell therapy and severe CRS.

Following target recognition, the chimeric amphotropic RNA vector encoding an anti-cyclin G1 construct or two killer genes (an anti-cyclin G1/HSV-tk) 500 (e.g., DNG64-CAR-V™ and DNR79-CAR-V™ with a SIG-binding peptide 501 and a gp70 env protein 502) enter the target cells and disrupt cell cycle progression by arresting cells in the G1 phase of the cell division cycle 600, ultimately inducing apoptosis in proliferating cancer cells 601. These vectors not only exert their therapeutic effects on the tumor, but also on the immune cells within the TME, thereby reducing the severity of CRS. The vectors invade rapidly proliferating T cells and activated macrophages to integrate their viral genome.

Importantly, these vectors modulate immune cell activity, reducing the severity of CRS without eliminating the efficacy of immunotherapy, e.g., CAR-T cell therapy, in contrast to the use of corticosteroids which lead to a reduction in the number or an elimination of engrafted CAR-T cells. Unlike steroids, the CAR-V™ vectors only target the CAR-T cells that are actively dividing at a given time at the site of injured tissues where collagenous SIG proteins are exposed. Thus, the CAR-T cells that are not actively dividing at the time of treatment are preserved.

The administration of the DNG64-CAR-V™ and DNG79-CAR-V™ vectors to patients experiencing CRS during an immunotherapy, e.g., CAR-T cell therapy, has three key effects. First, the administration of the CAR-V™ vectors enhances the efficacy of the immunotherapy by mitigating excessive cytokine production and release. Additionally, it contributes to tumor burden reduction both through efficacy of the CAR-T therapy and the anti-tumor activity of the DNG64-CAR-V™ and DNG79-CAR-V™ vectors. Furthermore, the administration of these CAR-V DNG64-CAR-VTM vectors advantageously reduces or eliminates the need for high dose steroid therapy.

What is claimed is:

1. A method of preventing, treating or lessening the effects of cytokine release syndrome (CRS) or an associated disorder in a patient, the method comprising administering a therapeutically effective amount of a chimeric amphotropic RNA vector encoding an anti-cyclin G1 construct or two killer genes (an anti-cyclin G1/HSV-tk) to the patient, wherein the patient has received, is receiving or about to receive an immunotherapy, wherein the immunotherapy is a Chimeric Antigen Receptor T cell (CAR-T) therapy, an antibody-based therapy, a Natural Killer cell (NK-cell) therapy, an Immune Checkpoint Inhibitors (ICI) therapy, Tumor-Infiltrating Lymphocytes (TIL) therapy, a cytokine therapy, a Bispecific T-cell Engagers (BiTEs) therapy, a neoTCR-T therapy or any other Adoptive Cell Transfer (ACT) therapy.

2. The method of claim 1, wherein the associated disorder is or comprises organ damage.

3. The method of claim 2, wherein the organ is selected from the group including but not limited to lung, kidney, liver, heart and brain.

4. The method of claim 1, wherein the chimeric amphotropic RNA vector is administered before the administration of the immunotherapy, at the same time as the administration of the immunotherapy, and/or after the administration of the immunotherapy.

5. The method of claim 1, wherein the therapeutically effective amount of a chimeric amphotropic RNA vector encoding an anti-cyclin G1 construct or two killer genes (an anti-cyclin G1/HSV-tk) is administered to the patient after the immunotherapy, when the immunotherapy is effective, functional, active, engrafted, eliciting a therapeutic response, or achieving immune activation.

6. The method of claim 1, wherein the chimeric amphotropic RNA vector is administered before the development of CRS, or symptoms thereof, and/or after the development of CRS, or symptoms thereof.

7. The method of claim 1, wherein the chimeric amphotropic RNA vector is administered before the onset of the CRS toxicity and/or after the onset of the CRS toxicity.

8. The method of claim 1, further comprising a step of determining the expression of a biomarker associated with CRS in the patient before and/or after administering a therapeutically effective amount of the chimeric amphotropic RNA vector.

9. The method of claim 8, wherein the biomarker comprises at least one of IL-6, IL-6R, C-reactive protein (CRP), IL-10, sIL-2R, TNF-α, lactate dehydrogenase (LDH), ferritin, G-CSF, sodium, creatinine, CD25, or CD69.

10. The method of claim 1, wherein the chimeric amphotropic RNA vector is administered to the patient until:
the serum biomarker levels decrease to levels observed in patients who do not develop CRS; or
the serum biomarker levels decrease to levels observed in the same patient before therapy treatment.

11. The method of claim 1, wherein the chimeric amphotropic RNA vector vector is administered at a dose of at least about 0.6×10¹⁰ RV copies.

12. The method of claim 1, wherein the chimeric amphotropic RNA vector is administered at a dose of from about 0.6×10¹⁰ RV copies to about 6×10¹⁰ RV copies.

13. The method of claim 1, comprising administering a dose of the chimeric amphotropic RNA vector to the subject daily for a period of from about 3 days to about 14 days after the administration of the immunotherapy, or from about 7 days to about 28 days after the administration of the immunotherapy.

14. The method of claim 1, comprising several administrations of the chimeric amphotropic RNA vector.

15. The method of claim 1, wherein the chimeric amphotropic RNA vector is administered once a day or multiple times a day.

16. The method of claim 1, wherein the chimeric amphotropic RNA vector is administered intravenously at a controlled rate.

17. The method of claim 1, further comprising a step of determining the levels of CAR-T cells circulating in the patient's blood stream after administering a therapeutically effective amount of the chimeric amphotropic RNA vector or upon completion of the treatment protocol.

18. The method of claim 1, wherein the patient has been diagnosed with cancer.

19. The method of claim 1, wherein the patient has mild or severe CRS induced by the CAR-T therapy.

20. The method of claim 1, further comprising a step of administering a therapeutically effective amount of a steroid therapy.
